(19) United States
(12) Patent Application Publication
(10) Pub. No.: US 2020/0388395 A1
(43) Pub. Date: Dec. 10, 2020

(54) APPARATUS, METHOD, AND NON-TRANSITORY COMPUTER-READABLE STORAGE MEDIUM

(71) Applicant: CANON KABUSHIKI KAISHA, Tokyo (JP)

(72) Inventors: Kohtaro Umezawa, Tokyo (JP); Kiyohide Satoh, Kawasaki-shi (JP)

(21) Appl. No.: 16/890,836

(22) Filed: Jun. 2, 2020

(30) Foreign Application Priority Data
Jun. 6, 2019 (JP) 2019-106573

(51) Int. Cl. G16H 50/20; G06N 20/00; G06N 7/00; G16H 30/20; G16H 30/40; G16H 40/20; G06Q 10/06; G16H 50/30

(52) U.S. Cl. CPC G16H 50/20; G06N 20/00; G06N 7/005; G16H 30/20; G16H 50/30; G16H 40/20; G06Q 10/06398; G06Q 10/063114; G16H 30/40

(57) ABSTRACT

An apparatus according to an aspect of the embodiments includes an acquisition unit configured to acquire medical image data, an inference unit configured to perform an inference with respect to the acquired medical image data, a calculation unit configured to calculate reliability based on a result of the inference, and a determination unit configured to determine a number of specialists who perform medical image data interpretation, based on the reliability.

### BACKGROUND OF THE INVENTION

### Field of the Invention

The aspect of the embodiments relates to an information processing apparatus that determines the number of specialists for medical image data interpretation based on an inference result, a method, and a non-transitory computer-readable storage medium.

### Description of the Related Art

In the medical field, specialists, such as doctors, use medical image data captured by various modalities (image capturing apparatuses) to perform diagnosis. The image capturing apparatuses include, for example, an ultrasonic wave diagnosis apparatus, a photo-acoustic image capturing apparatus (hereinafter referred to as photo-acoustic tomography (PAT) apparatus), and a magnetic resonance video apparatus (hereinafter referred to as magnetic resonance imaging (MRI) apparatus). In addition, other apparatuses, such as a computed tomography (CT) apparatus (hereinafter referred to as X-ray CT apparatus), and an optical coherence tomography apparatus (hereinafter referred to as OCT apparatus), are used as image capturing apparatuses. Because of enhanced performance of the image capturing apparatuses and increase in the number of times of capturing images, there has been a shortage of specialists who interpret medical image data with respect to an amount of data to be interpreted by the specialists.

In relation to a shortage of the specialists for medical image data interpretation, a technology to assist in the workflow of the specialists by a computer has been developed actively. Japanese Patent Application Laid-Open No. 2017-225542 discusses a technology by which an expression for calculating a certainty factor of an inference result in a region suspected of a lesion can be easily adjusted at medical image data interpretation, to reduce a burden of specialists, such as radiologists. Japanese Patent Application Laid-Open No. 2002-329190 discusses a technology of determining to whom medical image data interpretation is assigned, based on characteristics of each specialist and a degree of difficulty in identifying a lesion.

In the method discussed in Japanese Patent Application Laid-Open No. 2017-225542 and the method discussed in Japanese Patent Application Laid-Open No. 2002-329190, the number of specialists for medical image data interpretation has been determined in advance and changing the number are not taken into consideration.

### SUMMARY OF THE INVENTION

According to an aspect of the embodiments, an apparatus includes an acquisition unit configured to acquire medical image data, an inference unit configured to perform an inference with respect to the acquired medical image data, a calculation unit configured to calculate reliability based on a result of the inference, and a determination unit configured to determine a number of specialists who perform medical image data interpretation, based on the reliability.

Further features of the disclosure will become apparent from the following description of exemplary embodiments with reference to the attached drawings.

### BRIEF DESCRIPTION OF THE DRAWINGS

FIG. 1 is a schematic diagram illustrating a configuration of a medical image data display system according to a first exemplary embodiment.

FIG. 2 is a block diagram illustrating a configuration of a determination unit for the number of specialists for medical image data interpretation according to the present exemplary embodiment.

FIG. 3 is a diagram illustrating a relationship among reliability, a level of proficiency of a specialist, and the number of specialist for medical image data interpretation according to the present exemplary embodiment.

FIG. 4 is a diagram illustrating a relationship among reliability, a degree of difficulty of medical image data interpretation, and the number of specialists for medical image data interpretation according to the present exemplary embodiment.

FIG. 5 is a block diagram illustrating a computer and its peripheral devices according to the present exemplary embodiment.

FIG. 6 is a flowchart illustrating processing procedures of an information processing apparatus according to the present exemplary embodiment.

FIG. 7 is a flowchart illustrating processing procedures of an information processing apparatus according to a second exemplary embodiment.

### DESCRIPTION OF THE EMBODIMENTS

Exemplary embodiments to carry out the disclosure will be described below with reference to the accompanying drawings. The same constituent element is denoted by the same reference sign, and the redundant description thereof is omitted.

An information processing apparatus 10 according to a first exemplary embodiment, which has learned medical image data in advance, performs an inference using an inference unit 3 to analyze new medical image data at the time of acquiring the new medical image data. A reliability calculation unit 4 calculates reliability of the acquired inference result. Based on the calculated reliability, a number determination unit 5 determines the number of specialists for interpretation of the medical image data. The present exemplary embodiment will be described using the following example: An X-ray CT image of a chest region is acquired as medical image data, and whether lung cancer is included in the medical image data is inferred. In a case where the reliability of the inference is a threshold value or more, the number of specialists for image data interpretation is determined to be one. When the reliability of the inference is less than the threshold value, the number is determined to be two. The relationship between the number and the reliability is the one that determines the number based on the inference result, and therefore the relationship between the number and the reliability is not limited to the present exemplary embodiment.

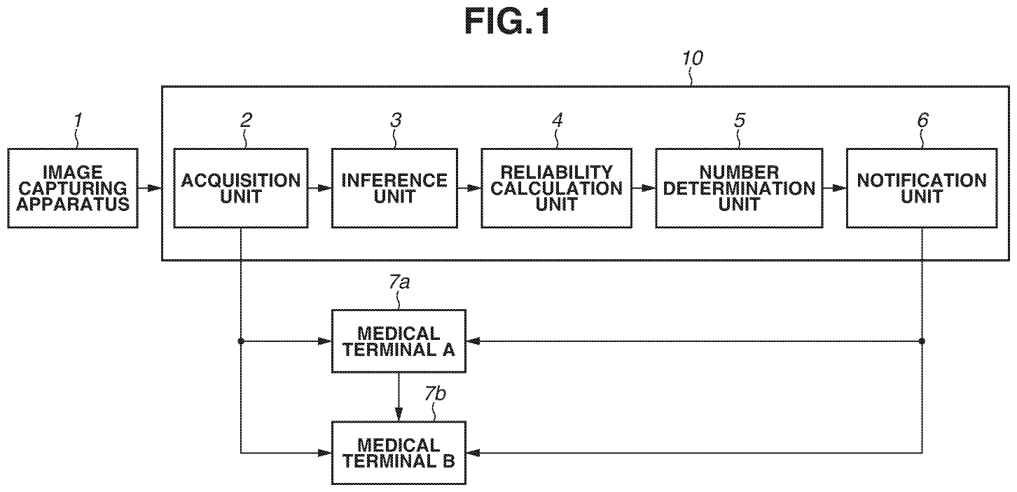

A configuration of the information processing apparatus 10 according to the present exemplary embodiment will be described below with reference to FIG. 1. FIG. 1 is a block diagram schematically illustrating an entire system including the information processing apparatus 10. The information processing apparatus 10 according to the present exemplary embodiment includes an image capturing apparatus 1, an acquisition unit 2, and the inference unit 3. The acquisition unit 2 acquires medical image data captured by the image capturing apparatus 1. The inference unit 3 performs an inference on the medical image data acquired by the acquisition unit 2. The information processing apparatus 10 further includes the reliability calculation unit 4 and the number determination unit 5. The reliability calculation unit 4 calculates the reliability based on a result of the inference performed by the inference unit 3. The number determination unit 5 determines the number of specialists for medical image data interpretation, based on the reliability calculated by the reliability calculation unit 4. Then, a notification unit 6 notifies a specialist(s) corresponding to the number determined by the number determination unit 5 of the medical image data acquired by the acquisition unit 2 on a medical terminal A (7a in FIG. 1) and a medical terminal B (7b in FIG. 1). In the present exemplary embodiment, the medical image data acquired by the acquisition unit 2 is captured by the image capturing apparatus 1. The number of medical terminals, on which the notification unit 6 performs notification, can be one or more.

A system configuration of the information processing apparatus 10 according to the present exemplary embodiment will be described below.

### (Image Capturing Apparatus 1)

The medical image data captured by the image capturing apparatus 1 is not limited to the X-ray CT image of the chest region. The medical image data may be, for example, an X-ray CT image of another region, a plain X-ray image, a scintigraphic image, a magnetic resonance (MR) image, a positron emission tomography (PET) image, single photon emission computed tomography (SPECT) image, an ultrasound image, an angiographic image, an endoscopic image, a thermographic image, an image for microscopic examination, and an ultrasonic light image. The target medical image data is not specifically limited, and the medical image data may be, for example, an electroencephalogram, a magnetoencephalogram, a biological image of a living object or an animal, an image of a surface of a biological body including a human body captured by a still camera or a video camera.

### (Acquisition Unit 2)

The acquisition unit 2 acquires medical image data to be a target of inference. As a method of acquiring the medical image data, a user may designate a medical image data file via a graphical user interface (GUI) using an input unit (not illustrated) included in the information processing apparatus 10 to cause the input unit to read the medical image data, or the information processing apparatus 10 may automatically acquire the medical image data based on an image capturing order transmitted from, for example, a hospital system.

The acquisition unit 2 may further acquire information about a threshold value or the like input by the user or acquire a parameter of a threshold value or the like set in advance, and the acquired information is used in processing in the subsequent units, such as inference unit 3 and the reliability calculation unit 4. The threshold value may be input by the user as appropriate, or set in advance. A threshold value adjustment unit 51 for adjusting the threshold value may be further included. That is, the information processing apparatus 10 as a medical image data processing apparatus according to the aspect of the embodiments further includes the threshold value adjustment unit 51 that adjusts the threshold value. The adjustment of the threshold value described below may be performed by the threshold value adjustment unit 51.

A storage medium that stores a program is a non-transitory storage medium. The acquisition unit 2 is not limited to a configuration of one storage medium, and may include a plurality of storage media.

### (Inference Unit)

The inference unit 3 analyzes medical image data acquired by the acquisition unit 2 to perform an inference on the medical image data. The inference unit 3 may perform the inference using a known deep neural network,

such as a convolutional neural network (CNN), targeting the medical image data input by the acquisition unit 2. Learning by an inference device may be newly performed on the network or may be based on the learned deep neural network. Alternatively, the inference unit 3 may perform the inference using calculation formulas or the like stored in another memory or the like.

The inference that is performed on the medical image data indicates that the deep neural network, which has completed learning for an input image, detects a target lesion in the input image. In the present exemplary embodiment, the medical image data serving as a target of medical image data interpretation is input to the deep neural network that has completed learning for detecting, for example, lung cancer. The learned deep neural network performs detection of a cancer region in the input medical image data. What the inference unit 3 performs the inference about is not limited to the present exemplary embodiment. For example, a local region including cancer in the medical image data may be delineated based on the inference performed by the inference unit 3. The inference unit 3 may calculate, for example, a possibility of inclusion of cancer in a local region in the medical image data, and a possibility of inclusion of cancer in the medical image data. The aspect of the embodiments can be applied to other lesions, such as pneumonia, emphysema, pneumothorax, and chronic obstructive pulmonary disease (COPD), and a target part can be organs other than a lung and tissues of muscles and bones. That is, the inference unit 3 is characterized by inferring a possibility of inclusion of a lesion in the medical image data. Alternatively, the inference unit 3 is characterized by inferring a possibility of inclusion of a lesion in a local region of the medical image data.

### (Reliability Calculation Unit 4)

The reliability calculation unit 4 calculates reliability based on an inference result from the inference unit 3. The reliability is an index indicating how reliable the inference result calculated by the deep neural network is.

The reliability includes reliability of an inference result on whether a disease is included in the whole of the medical image data, reliability of an inference result on a selected local region (such as result of classifying a plurality of diseases), and reliability of an inference result on a plurality of local regions. The reliability may be an average, a minimum value, or a maximum value of reliability of inference results on a plurality of local regions. In a case where the inference results are acquired based on multivalued classification, the reliability may be calculated with respect to a maximum value or a minimum value of the inference results. Alternatively, the reliability may be a maximum value or a minimum value of the reliability of the acquired inference results.

In the present exemplary embodiment, the reliability is calculated in accordance with the following procedures. In a case where the inference unit 3 performs deep neural network learning for binary classification, i.e., whether lung cancer is included in the whole of the medical image data or not, an output layer of the deep neural network is set as a softmax function. With the softmax function set in the output layer, a probability from 0 to 1 (referred to as softmax value) is calculated from input medical image data. In the binary classification problem, it can be considered that the farther the softmax value is away from 0.5 (reference value), reliability increases. Thus, how far the softmax value away from 0.5 (reference value) is expressed on percentage. That is, the reliability in the case of the binary classification is calculated based on the following Expression (1). In the Expression (1), K is the number of classes, and S is a softmax value. For example, when a softmax value acquired from the input medical image data is 0.99, reliability C based on the inference performed on the medical image data can be calculated as 10.99−0.51×100/0.5=98%. Meanwhile, when a softmax value acquired from the input medical image data is 0.45, reliability C by the inference performed on the medical image data can be calculated as 10.45-0.51×100/0.5=10%.

In a three or more class classification problem, e.g., a multivalued classification having K types of classes, a reference value can also be calculated by 1/K, and reliability C with respect to the softmax value S acquired from the medical image data can also be calculated using the reference value by the following Expression (1):

C=|S−1/K|×100/(1−1/K) (1).

A description will be given of a case where a local region in medical image data is classified into five types, for example, lung cancer, pneumonia, emphysema, pneumothorax, and COPD. When the local region is classified into five values, the reference value described above is expressed as 1/K=0.2, and therefore when the softmax value is 0.2 or more, the reliability calculated by the Expression (1) is defined as 0 to 100%. Meanwhile, when the softmax value is less than 0.2 (in this case, S=0), the reliability is calculated as |0−0.2|×100/(1−0.2)=25%. Reliability calculation is not limited to the calculation by the Expression (1). For example, the Softmax value, which is the inference result from the inference unit 3 may be used as the reliability. The larger the number of classes in a classification problem is, the reference value to be compared with the softmax value in the reliability calculation becomes smaller. In this case, for example, the softmax values of the respective classes included in the inference result may be compared with one another, and a value relative to other classes may be factored in as the reliability. When the softmax value is more than 0.5, the softmax value may be set as the reliability. When the softmax value is less than 0.5, a value obtained by adding a difference between 0.5 and the softmax value to 0.5 may be set as the reliability. That is, the reliability calculated by the reliability calculation unit 4 is characterized by being calculated based on the softmax value out of the inference result from the inference unit 3.

### (Number Determination Unit 5)

The number determination unit 5 determines the number of specialists for medical image data interpretation, based on the reliability calculated by the reliability calculation unit 4.

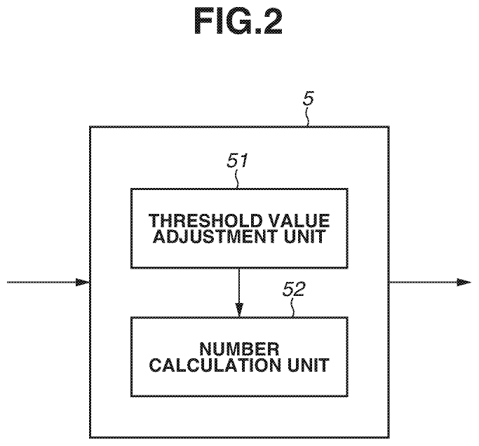

In a case where the calculated reliability is more than a set threshold value, the number determination unit 5 allocates the number smaller than the number that is allocated in a case where the calculated reliability is less than the set threshold value. On the other hand, in a case where the calculated reliability is less than the set threshold value, the number determination unit 5 determines the number larger than the number that is allocated in a case where the calculated reliability is more than the set threshold value. That is, the number determination unit 5 is characterized by determining the number of specialists for medical image data interpretation based on at least the reliability and the threshold value. More specifically, the threshold value serving as a target of comparison with the reliability may be set by the user on a graphical user interface (GUI), which is not illustrated, with respect to the reliability. A configuration of the number determination unit 5 will be described below with reference to FIG. 2. The number determination unit 5 includes the threshold value adjustment unit 51 that adjusts a threshold value based on threshold value data input by the user and a result calculated by the reliability calculation unit 4. A number calculation unit 52 calculates the number of specialists in medical image data interpretation based on the threshold value set by the threshold value adjustment unit 51. While the number determination unit 5 has a function of determining the number based on the reliability calculated by the reliability calculation unit 4, the configuration of the number determination unit 5 other than the function of determining the number is not limited to the present exemplary embodiment.

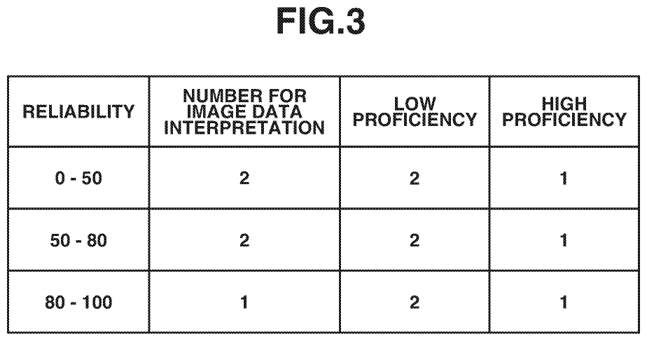

The threshold value that is set by the user may be set to separate high degrees and low degrees of reliability from each other. For example, when the reliability is low, the number can be determined to be two. When the reliability is high, the number can be determined to be one. A description will be given of a case where the user sets the threshold value, in detail below with reference to FIG. 3.

FIG. 3 is a diagram illustrating a table in which the calculated reliability and the number depending on the reliability are stored in a corresponding manner. In a case where the threshold value is set at 80% by the user and when the reliability is 80% or more, the number is determined to be one. When the reliability is less than 80%, the number is determined to be two. The number may be set different, for example, in accordance with another factor, in addition to the reliability calculated based on the inference result by the inference unit 3. For example, in the case illustrated in FIG. 3, the number determination unit 5 changes the number depending on a status of, for example, a degree of proficiency of a specialist, in addition to the reliability. When a skilled specialist (with high degree of proficiency) is in charge of medical image data interpretation, the number determination unit 5 assigns the medical image data interpretation to the specialist alone even if reliability calculated by the reliability calculation unit 4 is low. Meanwhile, when an unskilled specialist (with low degree of proficiency) is in charge of medical image data interpretation, the number determination unit 5 assigns the medical image data interpretation to two specialists even if reliability calculated by the reliability calculation unit 4 is high. The degree of proficiency may be quantitatively calculated based on an objective index, and may be input by the user.

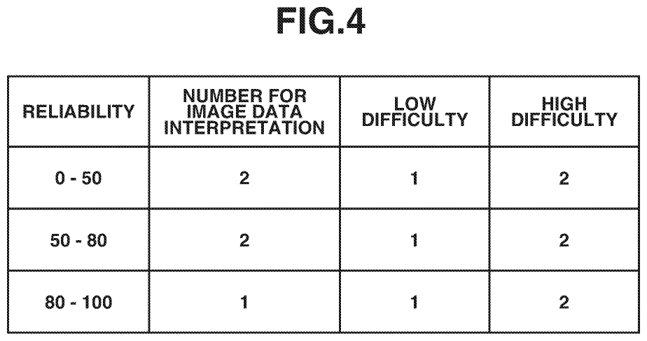

For example, in the case illustrated in FIG. 4, the number determination unit 5 further changes the number depending on a degree of difficulty in medical image data interpretation, in addition to reliability. According to the present exemplary embodiment, the number determination unit 5 determines the number by further factoring in information about a lesion and a disease having a high degree of difficulty in medical image data interpretation. For example, when a degree of difficulty in medical image data interpretation is high, the number determination unit 5 determines the number to be two even if the calculated reliability is high. When a degree of difficulty in medical image data interpretation is low, the number determination unit 5 determines the number to be one even if the calculated reliability is low.

The determination of the number based on reliability and other factors illustrated in FIGS. 3 and 4 is expected to produce effects of reducing errors in consideration of the burden of specialists in medical image data interpretation. That is, the number determination unit 5 determines the number based on at least either one of a degree of proficiency and a degree of difficulty in medical image data interpretation. The threshold value adjustment unit 51 may be in charge of adjusting the threshold value. The present exemplary embodiment is not limited to a configuration of determining the number based only on a degree of proficiency and a degree of difficulty. The number determination unit 5 may determine the number depending on reliability, and further depending on both a degree of proficiency and a degree of difficulty, or depending on other factors. Other factors include, for example, a degree of urgency and a degree of progression of a target lesion or disease. For example, when a degree of urgency is high even with high reliability, early and proper medical image data interpretation may be needed.

The present exemplary embodiment may have a configuration of changing a threshold value for determining the number based on, for example, a degree of proficiency and a degree of difficulty even when a relationship between reliability and other factors has not been defined in advance. Examples in which the threshold value adjustment unit 51 changes a threshold value include a configuration of setting the threshold value at 70% when one or two skilled specialists are in charge of the medical image data interpretation, and setting the number at one when the reliability is 70% or more and setting the number at two when the reliability is less than 70%. Meanwhile, the threshold value adjustment unit 51 may set the threshold value at 90% when one or two unskilled specialists are in charge of the medical image data interpretation, and set the number at one when the reliability is 90% or more and set the number at two when the reliability is less than 90%. In this case, the threshold value adjustment unit 51 may automatically change the predetermined threshold value in response to input of a name of a specialist to a computer. The acquisition unit 2 may acquire a degree of proficiency and a degree of difficulty. A degree of proficiency is determined based on, for example, a length of services in a target field, an error rate, and the number of times of medical image data interpretation. A degree of difficulty is determined based on, for example, an error rate per lesion or disease. Such information may be stored in a corresponding manner with an identification (ID) of each individual so that the number of specialists may be determined and the threshold value for determining the determination may be changed based on the information. That is, the information processing apparatus 10 according to the aspect of the embodiments is characterized by having the acquisition unit 2 that acquires medical image data, and the inference unit 3 that performs an inference on the medical image data acquired by the acquisition unit 2. The information processing apparatus 10 is characterized by further having the reliability calculation unit 4 that calculates reliability based on an inference result from the inference unit 3, and the number determination unit 5 that determines the number of specialists for medical image data interpretation based on the reliability. The number determination unit 5 transmits the determined number to the subsequent unit which is the notification unit 6.

### (Notification Unit 6)

The notification unit 6 executes, for example, processing to determine a specialist(s) to whom a request for medical image data interpretation is performed, based on the number determined by the number determination unit 5, and processing to notify the determined specialist(s) of medical image data. That is, the information processing apparatus 10 according to the aspect of the embodiments further includes the notification unit 6 that performs notification of the medical image data based on the number determined by the number determination unit 5. The notification unit 6 determines a destination to which a request for medical image data interpretation is performed, based on the number determined by the number determination unit 5. The destination to which the request for medical image data interpretation is performed includes, for example, the medical terminal A (7a) and the medical terminal B (7b) each corresponding to a different one of specialists. The contents of the notification may be different depending on a degree of reliability. The notification unit 6 may transmit medical image data to a specific specialist when the reliability is less than the threshold value, while the notification unit 6 may transmit medical image data to an application for a conference when the reliability is more than the threshold value. Alternatively, when the reliability is less than the threshold value, the notification unit 6 may transmit the medical image data to another artificial intelligence (AI) and cause the AI to perform an inference for further examination of the case. Another AI may be, for example, a deep neural network that has learned to detect other lesions and diseases, a deep neural network having a different number of layers and a different structure, and an AI that performs an inference based on a statistical method.

The notification unit 6 may cause a display unit, which is not illustrated, to display the determined number and perform the notification. Consequently, the notification unit 6 may allow the user to check and to be aware of the number as a result of an inference performed on the input medical image data. The present exemplary embodiment may have a configuration in which the notification unit 6 can check a job status of the notified destination to which the request for medical image data interpretation has been performed. That is, the notification unit 6 is characterized by performing further notification of a job status of the notified specialist in the medical image data. The notification unit 6 can perform notification to all appropriate destinations to which a request for medical image data interpretation is performed without omission, for example, by acquiring information about whether the transmitted medical image data or file has been opened, or information about whether a report shows that some kind of action has been taken.

### (Medical Terminal A (7a) And Medical Terminal B (7b))

The medical terminal A and the medical terminal B are terminals each corresponding to a different one of specialists determined by the notification unit 6. For example, the medical terminal A (7a) corresponds to a specialist A and the medical terminal B (7b) corresponds to a specialist B. The medical terminals receive information including the medical image data notified by the notification unit 6 and display the medical image data to the specialists. The medical terminal A (7a) and the medical terminal B (7b) may be independent from each other and communicate with each other. The number of the medical terminals can be one or more.

An arithmetic circuit that is used for the acquisition unit 2, the inference unit 3, the reliability calculation unit 4, the number determination unit 5, and the notification unit 6 included in the information processing apparatus 10 may be a dedicatedly designed workstation. Elements of the arithmetic circuit may be configured by different hardware. At least part of the elements of the arithmetic circuit may be configured by single hardware. That is, each unit included in the information processing apparatus 10 is configured by a processor such as a central processing unit (CPU) and a graphics processing unit (GPU), and an arithmetic circuit such as a field programmable gate array (FPGA) chip. These units may be configured not only by a single processor and a single arithmetic circuit but also by a plurality of processors and a plurality of arithmetic circuits.

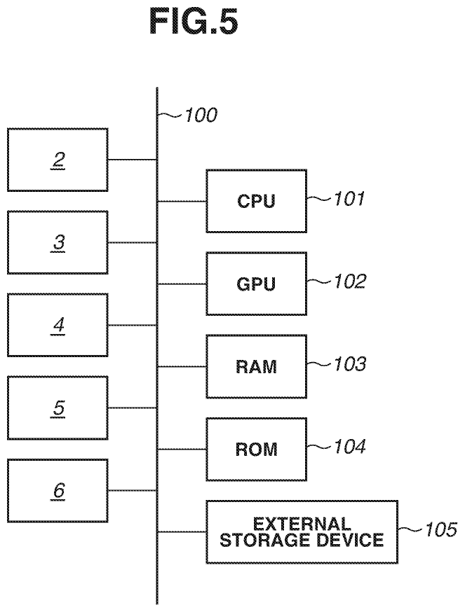

FIG. 5 illustrates a detailed configuration of the arithmetic circuit for the acquisition unit 2, the inference unit 3, the reliability calculation unit 4, the number determination unit 5, and the notification unit 6. The arithmetic circuit for the acquisition unit 2, the inference unit 3, the reliability calculation unit 4, and the number determination unit 5, and the notification unit 6 includes a CPU 101, a GPU 102, a random-access memory (RAM) 103, a read-only memory (ROM) 104, and an external storage device 105, and these elements are connected via a system bus 100. A liquid crystal display serving as a display unit (not illustrated), and a mouse and keyboard serving as an input unit (not illustrated) may be connected to the acquisition unit 2, the inference unit 3, the reliability calculation unit 4, and the number determination unit 5, and the notification unit 6.

The acquisition unit 2, the inference unit 3, the reliability calculation unit 4, and the number determination unit 5, and the notification unit 6 may serve as an on-premise system, or may serve as a program on a network, such as a server and a cloud-based system, to execute the processing.

The elements of the information processing apparatus 10 may be individual devices, or may be integrated as one device. Alternatively, at least part of the elements of the information processing apparatus 10 may be integrated as one device.

### (Number Determination Procedure)

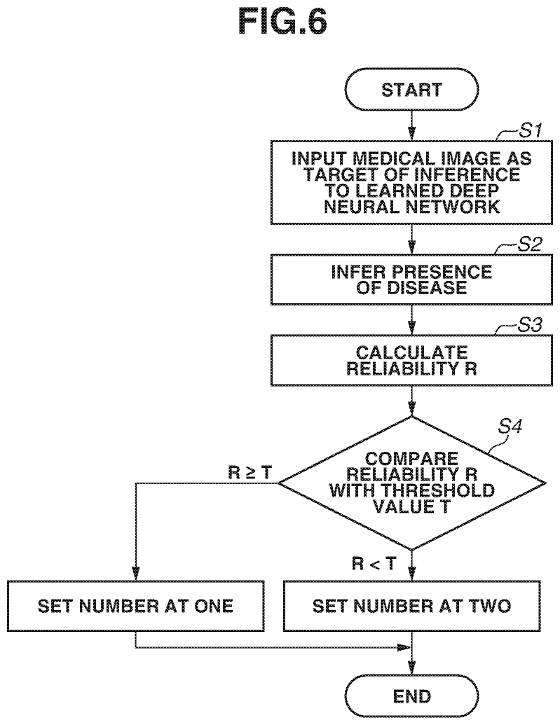

FIG. 6 is a flowchart for determining the number of specialists for medical image data interpretation to be one or two by setting one threshold value for determining the number with respect to reliability. A description will be given of the processing procedure of, for example, setting a threshold value T (e.g., 95%) to reliability R that has been predetermined in an application by the user or the like, setting the number at one when the reliability R is the threshold value T or more, and setting the number at two when the reliability R is less than the threshold value T. The flowchart starts in a state where a deep neural network that detects presence of a disease from the whole of a chest CT image has already learned data. In step S1, the acquisition unit 2 inputs a chest CT image to the deep neural network. In step S2, the inference unit 3 performs an inference of, for example, presence of a disease in the whole of the chest CT image on the input medical image data. In step S3, the reliability calculation unit 4 calculates the reliability R based on a result of the inference. In step S4, the number determination unit 5 compares the calculated reliability R and the threshold value T to determine the number of specialists for medical image data interpretation. Specifically, in a case where the calculated reliability R is the threshold value T or more, the number determination unit 5 determines the number to be one. In a case where the calculated reliability R is less than the threshold value T, the number determination unit 5 determines the number to be two. According to the present exemplary embodiment, the number determination unit 5 can determine the number of specialists who perform medical image data interpretation, based on the reliability of the inference performed by the inference unit 3 on the medical image data. This can reduce the possibility of oversight and a diagnostic error at medical image data interpretation even when reliability obtained by the inference is low.

### First Modification of First Exemplary Embodiment

While in the first exemplary embodiment, the number is determined by calculating reliability to the inference result from the deep neural network, the aspect of the embodiments is not limited to the case of using the deep neural network. For example, the number may be determined by calculating reliability to an inference result calculated by machine learning, such as a support vector machine (SVM) other than the deep neural network or other known methods.

### Second Modification of First Exemplary Embodiment

While the first exemplary embodiment has been described with reference exclusively to lung cancer, the aspect of the embodiments is not limited to the lung cancer and may be applied to other kinds of cancer and disease.

### Third Modification of First Exemplary Embodiment

While the first exemplary embodiment has been described using the example in which reliability is defined on percentage by how far the softmax value is away from 0.5 in the case of the binary classification problem, the exemplary embodiments of the disclosure are not limited thereto. For example, a value of 1/K (K is the number of classes) is referred to as a reference value for reliability, and the following conditions may be defined by dividing cases depending on whether a softmax value S of input medical image data is the reference value or more, or less than the reference value. With such definition, reliability can be calculated from 0 to 100% even when the softmax value S is a value of 1/K or more, or less than the value of 1/K.

C=(S−1/K)×100/(1−1/K) (where 1≥S≥1/K)

C=(1/K−S)×100/(1/K−0)) (where S<1/K) (2)

Other calculation methods for reliability include a method in which the reliability calculation unit 4 executes clustering based on an inference result from the deep neural network and compares distributions to calculate reliability.

First, the information processing apparatus 10 acquires a class label (referred to as classification label), into which the medical image data is classified by input of the medical image data to the learned deep neural network. The classification label serving as an inference result is, for example, a softmax value. The learned deep neural network outputs softmax values to the respective classification classes as inference results so that a total of the softmax values becomes 1. The information processing apparatus 10 compares the softmax values output to the respective classes, and presumes that the target medical image data is classified into a class having the highest softmax value. Subsequently, the reliability calculation unit 4 uses a distance between classes to calculate reliability as follows, for example. First, the reliability calculation unit 4 calculates a class distribution serving as the centroid of the presumed class, from class distributions of softmax values in medical image data, other than the target medical image data, belonging to the presumed class. With respect to other classes, the reliability calculation unit 4 calculates class distributions serving as the centroids of respective classes, from class distributions of softmax values in the medical image data belonging to the respective classes. Using the class distribution serving as the centroid of the softmax value of the presumed class and the class distributions serving as the centroids of the softmax values of the other classes, the reliability calculation unit 4 calculates a mean-square distance between the class distribution of the Softmax value of the target medical image data and each of the class distributions serving as the centroids of all the classes. The reliability calculation unit 4 calculates a value of adding the mean-square distance between the class distribution of the target medical image data and the class distribution serving as the centroid of the presumed class to a mean-square distance between the class distribution of the target medical image data and a class distribution serving as the centroid of a next closest class. The reliability is a value that is obtained by dividing the mean-square distance between the class distribution of the target medical image data and the class distribution serving as the centroid of the next closest class by the added value described above, and that is represented on percentage. In this manner, the reliability can be calculated on the strictest condition between the class distribution of the target medical image data and the class distribution serving as the centroid of the next closest class.

The reliability calculation unit 4 may execute clustering based on softmax values, compare a class having the classification label with other classes, and calculate how close the input medical image data is to the classification label to set a resultant value as the reliability. The method of calculating the reliability by clustering by the reliability calculation unit 4 will be described below. First, the reliability calculation unit 4 calculates a distance A and a distance B. The distance A is a distance between the centroid of another class that is the closest to the input medical image data but does not belong to the class having the classification label and the input medical image data. The distance B is a distance between the centroid of the class having the classification label and the input medical image data. A value of (1−B/(A+B)−0.5)×100 may be calculated from the calculated value, where 0.5 is a reference value for the reliability between two classes. As a matter of course, the reference value may be changed depending on the number of classes, and may be a constant value.

Alternatively, the reliability calculation unit 4 compares the distance A, which is a distance between the centroid of the class having the classification label and the centroid of another class that is the closest to the input medical image data, and the distance B, which is a distance between the input medical image data and the centroid of the class having the classification label. That is, the reliability calculation unit 4 may calculate the reliability by comparing the distances between the input medical image data and the centroids of the two classes, as expressed by, for example, ((1−B/A)−0.5)×100, where 0.5 is a reference value for the reliability between the two classes.

As a method of calculating reliability by the reliability calculation unit 4, the reliability may be extracted by various methods using an accurate rate, an average value, and dispersion, and other related statistic values acquired from test data, evaluation data and the like. The calculation of the reliability by the reliability calculation unit 4 may use, other than the accurate rate, a learning error rate, a learning error value, an error rate with respect to the evaluation data, an error rate with respect to the test data, a loss function value, and an index as to whether over-learning has occurred. Further, it can be considered that the calculation of reliability may use accuracy, a degree of singularity that is a ratio of test positive data in disease data, sensitivity that is a ratio of test negative data in non-disease data, and a hit rate of positive reaction that is disease-affection data out of the test positive data. The calculation may use a hit rate of negative reaction that is non-disease affection data out of the test negative data, and a value related to a disease with respect to a threshold value in a receiver operating characteristic (ROC) curve or a free-response receiver operating characteristic (FROC) curve. For example, with an accurate rate of 90% with respect to test data when the deep neural network has learned medical image data, the reliability calculation unit 4 may set reliability at 90%. That is, the reliability calculation unit 4 is characterized by calculating reliability based on the accuracy of the inference performed by the inference unit 3.

Reliability may also be a value of a computed result, such as a Jaccard coefficient, a Dice coefficient, and a Simpson coefficient. In this case, with respect to a set X and a set Y, the Jaccard coefficient J is expressed as J=|X∩Y|/|X∪Y|, the Dice coefficient D is expressed as D=2|X∩Y|/(|X|+|Y|), and the Simpson coefficient S is expressed as S=|X∩Y|/min(|X|, |Y|).

### Fourth Modification of First Exemplary Embodiment

The calculation of the reliability is performed by the reliability calculation unit 4 determining the reliability based on the softmax value which is the inference result output from the learned deep neural network. As for a softmax value, when an inference device determines the presence of a class serving as a target of classification in target medical image data, a high value is obtained in the class. The present modification will be described using a case where the deep neural network used in the inference unit 3 infers a softmax value with respect to each of pixels of the target medical image data. With the present configuration, the output from the inference unit 3 is not a softmax value per medical image data, but each of pixels has a softmax value. In this case, the reliability calculation unit 4 may acquire an average of the softmax values in the whole of the medical image data for each class to use the average for the reliability calculation described above. Alternatively, the reliability calculation unit 4 may be configured to set a threshold value with respect to a magnitude of a softmax value, and factor in the number of pixels or the area of pixels that exceed the threshold value or do not exceed the threshold value as reliability. Alternatively, a gradient of a softmax value with respect to each pixel may be calculated. This configuration can increase expectations for advantageous effect that the user can grasp which pixel in the target medical image data contributes to the reliability calculation.

### Fifth Modification of First Exemplary Embodiment

The inference unit 3 described above has been described using the example of the deep neural network that performs detection of the presence of lung cancer, and the example of the deep neural network that classifies data into five classes. However, the configuration may generate an enormous number of classes serving as an inference target with respect to a single deep neural network, and a sufficient inference result may not be acquired when a correlation between the classes is weak. Thus, the aspect of the embodiments may have a plurality of deep neural networks to perform an inference. The inference unit 3 is configured by the deep neural networks, for example, corresponding to respective diseases or lesions, and each of the deep neural networks performs an inference. The reliability calculation unit 4 may calculate reliability based on each of the inference results, and the number determination unit 5 may determine the number. The number may be determined based on reliability with respect to each lesion and other values, such as a degree of proficiency and a degree of difficulty. For example, when a plurality of inferences with a high degree of difficulty is performed, it can be considered that a larger number of specialists are allocated. As a matter of course, a destination to which a request for medical image data interpretation is performed may be changed depending on an inference target of the deep neural network.

### Sixth Modification of First Exemplary Embodiment

The fifth modification has been described using the case, in which the deep neural networks used in the inference unit 3 output the respective inference results. In a sixth modification of the first embodiment, a description will be given of processing that is performed when a plurality of deep neural networks indicates mutually different inference results with respect to the target medical image data. An example of such case is that a deep neural network A, which performs an inference with respect to A, and a deep neural network B, which performs an inference with respect to B, perform inferences on an identical image region and obtain an inference result A and an inference result B, respectively, both with high softmax values. The identical image region may be the whole of the target image region, or may include an overlapping region in part of the target image region.

When the different deep neural networks indicate different inference results with high softmax values on the overlapping region, the reliability calculation unit 4 calculates reliability by multiplying each of the inference result A and the inference result B by a co-occurrence probability or a degree of similarity of the inference result A and the inference result B. With the present configuration, the number determination unit 5 can appropriately determine the number even when the deep neural network erroneously outputs a high softmax value. That is, when different inference results are obtained with respect to the medical image data having the identical region or the overlapping region in at least part thereof, the reliability calculation unit 4 is characterized by calculating the reliability based on at least either one of the co-occurrence probability and the degree of similarity of the inference results.

### Seventh Modification of First Exemplary Embodiment

The reliability calculation unit 4 calculates the reliability not only based on the magnitude of the softmax value in the inference result obtained using the deep neural network, but also by factoring in performance of the deep neural network itself in addition to the magnitude of the softmax value. For example, the softmax value output from the deep neural network having an accurate rate of 90% may be multiplied by the accurate rate of 90%, and a resultant value may serve as the reliability.

### Eighth Modification of First Exemplary Embodiment

When the reliability calculated based on the inference result from the inference unit 3 is less than the threshold value, the notification unit 6 notifies a plurality of specialists of the request for medical image data interpretation. The method of performing notification of the request for medical image data interpretation can be considered to have some variations. The variations include, for example, a case of performing the request for medical image data interpretation to the specialists in parallel. When the number of specialists to which the request for medical image data interpretation is performed is two, the notification unit 6 transmits the identical medical image data to the medical terminal A (7a) corresponding to the specialist A and the medical terminal B (7b) corresponding to the specialist B illustrated in FIG. 1, and requests medical image data interpretation. When the notification unit 6 requests medical image data interpretation in parallel, the specialists can perform medical image data interpretation themselves without receiving advice from others. Meanwhile, when medical image data interpretation results from the specialists (first specialist and second specialist) are different, it can be considered, for example, to seek judgment from a third person (third specialist) in light of the medical image data interpretation results. Alternatively, there may be a process of notifying a specialist of information about a medical image data interpretation result that is different from his/her own medical image data interpretation result and requesting the specialist for medical image data interpretation again. That is, the notification unit 6 is characterized by, when the medical image data interpretation result from the first specialist and the medical image data interpretation result from the second specialist are different from each other, notifying the third specialist of the medical image data, the medical image data interpretation by the first specialist, and the medical image data interpretation by the second specialist.

Meanwhile, there may be a case of requesting a plurality of specialist for medical image data interpretation in series (in a hierarchical way). In this case, for example, the specialist B performs medical image data interpretation in light of a result of medical image data interpretation performed by the specialist A, whereby reduction of a labor between the specialists can be expected. A description will be given of a case where the number of specialists to which the notification unit 6 performs the request for medical image data interpretation is two with reference to FIG. 1. When the number of specialists is two, the notification unit 6 notifies the specialist A (first specialist) of medical image data. Subsequently, the notification unit 6 notifies the specialist B (second specialist) of the medical image data and also a result of medical image data interpretation performed by the specialist A (first specialist). That is, when the number of specialists determined by the number determination unit 5 is two or more, the notification unit 6 is characterized by notifying the first specialist of the medical image data, and notifying the second specialist of the medical image data and also the result of the medical image data interpretation performed by the first specialist.

An information processing apparatus 10 according to a second embodiment acquires a brain MRI image as medical image data and infers whether a brain tumor is included in the medical image data. The present exemplary embodiment will be described using an example, in which two threshold values, a threshold value T1 and a threshold value T2 (T1>T2), are set. When reliability of inference is the threshold value T1 or more, the number of specialists is determined to be zero. When reliability of inference is the threshold value T2 or more and less than the threshold value T1, the number is determined to be one. When reliability of inference is less than the threshold value T2, the number is determined to be two.

A configuration of the second exemplary embodiment except for the number determination unit 5 and the notification unit 6 is the same as the configuration of the first exemplary embodiment, and thus a description will be given exclusively to part of the number determination unit 5 and the notification unit 6 that is different from the first exemplary embodiment.

### Number Determination 5

The number determination unit 5 determines information about the number of specialists who interpret medical image data based on the reliability calculated by the reliability calculation unit 4.

In the present exemplary embodiment, two threshold values, a threshold value T1 (e.g., 95%) and a threshold value T2 (e.g., 75%), are set. When reliability R is the threshold value T1 or more (R>T1), the number is determined to be zero. When reliability R is the threshold value T2 or more and less than the threshold value T1 (T1>R≥T2), the number is determined to be one. When reliability R is less than the threshold value T2, the number is determined to be two.

The present exemplary embodiment is not limited to the configuration in which the higher the reliability R is, the number of specialists is decreased, and a relationship between the reliability, the threshold values, and the number may be freely set. For example, the number is set at one when the reliability R is the threshold value T1 or more, and at two when the reliability R is the threshold value T2 or more and less than the threshold value T1. Further, the number is set at zero when the reliability R is less than the threshold value T2, and the medical image data may be transmitted to a different AI for inference. With this configuration, when the reliability is less than the threshold value T2, the medical image data can be transmitted to the different AI without medical image data interpretation and an inference result from the different AI can be referred.

The number may be set at three or more. For example, when the reliability R is the threshold value T1 or more, the number may be set at one. When the reliability R is the threshold value T2 or more and less than the threshold value T1, the number may be set at two. When the reliability R is less than the threshold value T2, the number may be set at three.

### (Notification Unit 6)

When the number determined by the number determination unit 5 is zero, the notification unit 6 does not notify a specialist of the medical image data. That is, the notification unit 6 is characterized by not performing notification of the medical image data, based on the number determined by the number determination unit 5. The processing executed when the number is determined to be other than zero is the same as that in the first exemplary embodiment.

A procedure of determining the number in the information processing apparatus 10 according the present exemplary embodiment will be described below.

### (Number Determination Procedure)

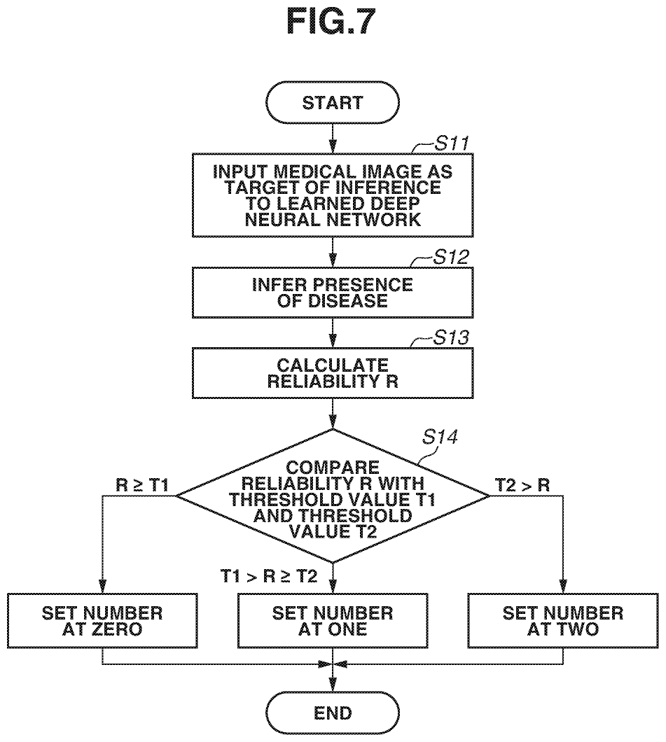

FIG. 7 is a flowchart for determining the number to be zero to two by setting two threshold values with respect to reliability. Assume that threshold values, a threshold value T1 (e.g., 97%) and a threshold value T2 (e.g., 60%, T1>T2), are set with respect to reliability R that has been predetermined in an application. The flowchart starts in a state where the deep neural network that performs detection of the presence of a disease from the whole of the brain MR image has already learned data. In step S11, the acquisition unit 2 inputs the brain MRI image to the deep neural network. In step S12, the inference unit 3 infers, for example, the presence of the disease in the whole of the brain MRI image with respect to the input medical image data. In step S13, the reliability calculation unit 4 calculates the reliability R based on a result of the inference. In step S14, the number determination unit 5 compares the calculated reliability R and the two threshold values T1 and T2 to determine the number of specialists. Specifically, the number determination unit 5 can determine the number to be zero when the reliability R is the threshold value T1 or more, to be one when reliability R is the threshold value T2 or more and less than the threshold value T1, and to be two when the reliability R is less than the threshold value T2.

According to the present exemplary embodiment, the number determination unit 5 can more flexibly determine the number based on the reliability of the inference with respect to the medical image data. This can reduce the possibility of oversight and a diagnostic error at the time of medical image data interpretation even when the reliability of the inference is low.

### First Modification of Second Exemplary Embodiment

While the second exemplary embodiment has been described with reference exclusively to the brain tumor, the aspect of the embodiments is not limited to the brain tumor and may be applied to other diseases.

### Second Modification of Second Exemplary Embodiment

While the second exemplary embodiment has been described with reference exclusively to the brain MRI image, the aspect of the embodiments is not limited to the brain MRI image and may be applied to other medical image data.

### Third Modification of Second Exemplary Embodiment

The modifications of the first exemplary embodiment may be applied to the second exemplary embodiment.

### OTHER EMBODIMENTS

Embodiment(s) of the disclosure can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the disclosure has been described with reference to exemplary embodiments, it is to be understood that the disclosure is not limited to the disclosed exemplary embodiments. The scope of the following claims is to be accorded the broadest interpretation so as to encompass all such modifications and equivalent structures and functions.

This application claims the benefit of Japanese Patent Application No. 2019-106573, filed Jun. 6, 2019, which is hereby incorporated by reference herein in its entirety.

What is claimed is:

1. An apparatus, comprising:
an acquisition unit configured to acquire medical image data;
an inference unit configured to perform an inference with respect to the acquired medical image data;
a calculation unit configured to calculate reliability based on a result of the inference; and
a determination unit configured to determine a number of specialists who perform medical image data interpretation, based on the reliability.

2. The apparatus according to claim 1, wherein the calculation unit is configured to calculate the reliability based on an index of how reliable a result of the inference is.

3. The apparatus according to claim 1, wherein the calculation unit is configured to calculate the reliability based on a softmax value out of a result of the inference.

4. The apparatus according to claim 1, wherein the determination unit is configured to determine the number based on at least the reliability and a threshold value.

5. The apparatus according to claim 4, wherein, in a case where the calculated reliability is less than the threshold value, the determination unit is configured to determine the number to be larger than the number that is determined in a case where the calculated reliability is more than the threshold value.

6. The apparatus according to claim 4, wherein, in a case where the calculated reliability is more than the threshold value, the determination unit is configured to determine the number to be smaller than the number that is determined in a case where the calculated reliability is less than the threshold value.

7. The apparatus according to claim 1, wherein the determination unit is configured to determine the number based on at least either one of a degree of proficiency of the specialists and a degree of difficulty in the medical image data interpretation.

8. The apparatus according to claim 1, further comprising a notification unit configured to perform notification of the medical image data based on the determined number.

9. The apparatus according to claim 8, wherein the notification unit is configured not to perform notification of the medical image data based on the determined number.

10. The apparatus according to claim 4, further comprising an adjustment unit configured to adjust the threshold value.

11. The apparatus according to claim 10, wherein the adjustment unit is configured to adjust the threshold value based on at least either one of a degree of proficiency of the specialists and a degree of difficulty in the medical image data interpretation.

12. The apparatus according to claim 8, wherein the notification unit is configured to perform further notification for the medical image data of a job status of a specialist to which the notification has been performed.

13. The apparatus according to claim 1, wherein, in a case where the inference unit obtains a plurality of different inference results with respect to medical image data having an identical region or an overlapping region in at least part thereof, the calculation unit is configured to calculate the reliability based on at least either one of a co-occurrence of the inference results and a degree of similarity of the inference results.

14. The apparatus according to claim 8, wherein in a case where the determined number is two or more, the notification unit is configured to notify a first specialist of the medical image data, and notify a second specialist of the medical image data and further a result of medical image data interpretation by the first specialist.

15. The apparatus according to claim 8, wherein, in a case where a result of the medical image data interpretation by a first specialist and a result of the medical image data interpretation by a second specialist are different from each other, the notification unit is configured to notify a third specialist of the medical image data, the result of the medical image data interpretation by the first specialist, and the result of the medical image data interpretation by the second specialist.

16. The apparatus according to claim 1, wherein the calculation unit is configured to calculate the reliability based on accuracy of the performed inference.

17. The apparatus according to claim 1, wherein the inference unit is configured to infer a possibility of inclusion of a lesion in the medical image data.

18. The apparatus according to claim 1, wherein the inference unit is configured to infer a possibility of inclusion of a lesion in a local region in the medical image data.

19. A method, comprising:
acquiring medical image data;
performing an inference with respect to the acquired medical image data;
calculating reliability based on a result of the inference; and
determining a number of specialists who perform medical image data interpretation, based on the reliability.

20. A non-transitory computer-readable storage medium storing a program for causing a computer to execute a process comprising:
acquiring medical image data;
performing an inference with respect to the acquired medical image data;
calculating reliability based on a result of the inference; and
determining a number of specialists who perform medical image data interpretation, based on the reliability.
